(19) United States
(12) Patent Application Publication
(10) Pub. No.: US 2025/0120929 A1
(43) Pub. Date: Apr. 17, 2025

(54) METHOD OF TREATING VIRUS-ASSOCIATED INFECTION USING ACETAMINOPHEN

(71) Applicant: Sanbiozi Medical Science Inc., Dover, DE (US)

(72) Inventor: Sheng-Kuang TSAI, Taipei City (TW)

(21) Appl. No.: 18/917,086

(22) Filed: Oct. 16, 2024

Related U.S. Application Data
(60) Provisional application No. 63/590,768, filed on Oct. 16, 2023.

(51) Int. Cl. A61K 31/167

(52) U.S. Cl. CPC A61K 31/167

(57) ABSTRACT

The present invention relates to methods of treating virus infection in a human patient or a mammal, wherein the methods comprise administration of a therapeutically effective amount of an acetaminophen, or a pharmaceutically acceptable salt thereof.

### CROSS REFERENCE TO RELATED APPLICATION

This application claims the benefit of the Provisional Application Ser. No. 63/590,768, filed on Oct. 16, 2023 by the present inventor.

### FIELD OF THE INVENTION

The present invention relates to methods of treating virus infection in a human patient or a mammal, wherein the methods comprise administration of a therapeutically effective amount of an acetaminophen, or a pharmaceutically acceptable salt thereof.

### BACKGROUND

Coronavirus (CoV) infection has become a global crisis since 2019. With the cooperative work, there are vaccines and antiviral drugs against COVID-19 are available currently. However, the reoccurring infections and breakthrough infections can be observed over time, and the mutated variants would soon emerge to threat the lives even though they have been vaccinated. That is, it is still imperative to develop different antiviral compound against such a CoV infection.

At an extremely coincidental timing, the COVID in 2019, we began to explore the antiviral function of “generic drugs”.

In addition to the CoV that has attracted the most attention recently, swine virus PRRS is also worthy of public attention. PRRS is abbreviated from “porcine reproductive and respiratory syndrome”, which was first recognized in the United States in 1987. It is already been widely disseminated and affects domestic pigs and the related economy. The symptoms include reproductive failure, pneumonia and increased susceptibility to secondary bacterial infection.

PRRS is the most economically significant disease to affect US swine production since the eradication of classical swine fever and costs the United States pig industry around $650 million annually, and recent estimates in Europe determined that it costs almost €1.5b every year.

“Generic drugs” can be developed more quickly because of their well-known drug safety. The purpose of the present invention is to find a “generic drug”, a known safe drug, which can effectively fight against viruses.

### SUMMARY OF THE INVENTION

In one aspect, provided herein is a method of treating virus infection in a subject. The method includes administering to the subject in need thereof a therapeutically effective amount of a pharmaceutical composition comprising an acetaminophen.

In some embodiments, the patient is an adult human patient. In other embodiments, the patient is a pediatric (i.e., infant, child or adolescent) human patient. A pediatric patient may be an individual from birth to up to 18-21 years of age. The patient may be experiencing a coronavirus-infection. In some embodiments, the subject is a piglet which receiving PRRS virus infection.

Other objects, features, and advantages of the present invention will be apparent to one of skill in the art from the following detailed description and figures.

### BRIEF DESCRIPTION OF THE DRAWING

FIG. 1 shows the cytotoxicity of acetaminophen on Mv1Lu cells after five days of incubation.

FIG. 2 shows the body weight of MHV-infected mice with acetaminophen treatment.

FIG. 3 shows the survival rate of MHV-infected mice with acetaminophen treatment.

### DETAILED DESCRIPTION OF THE INVENTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The term “comprise” or “comprising” is generally used in the sense of include/including which means permitting the presence of one or more features, ingredients or components. The term “comprise” or “comprising” encompasses the term “consists” or “consisting of.”

According to the present invention, an acetaminophen compound is acetaminophen, its derivatives, or its prodrug.

As used herein, the term “treating” refers to the therapeutic measures to a disease or the symptoms or conditions of a disease, which include but are not limited to applying or administering one or more active agents to a subject suffering from the disease or the symptoms or conditions of the disease or exacerbation of the disease. The purpose of the therapeutic measures is to treat, cure, mitigate, relieve, alter, remedy, ameliorate, improve, or affect the disease, the symptoms or conditions of the disease, disability caused by the disease, or exacerbation of the disease. Specifically, the present invention provides an acetaminophen compound for treating virus infection.

As used herein, the term “individual” or “subject” includes human or non-human animals, in particular mammal, for example, companion animals (such as dogs, cats and the like), farm animals (such as cattle, sheep, pigs, horses, etc.), or laboratory animals (such as rats, mice, guinea pigs, etc.).

As used herein, the term “effective amount” refers to the amount of an active ingredient achieving desired biological efficacy or therapeutic effects in a subject being treated, for example, pain relief.

### Examples

The present invention utilized the Mouse Hepatitis Virus (MHV) and swine virus PRRS as the infectious agents to evaluate the effectiveness of acetaminophen in vivo. We aimed to perform mouse and piglet survival experiments to completely assess acetaminophen against virus infection as a preclinical evaluation.

### In Vitro Test for Cytotoxicity of Acetaminophen

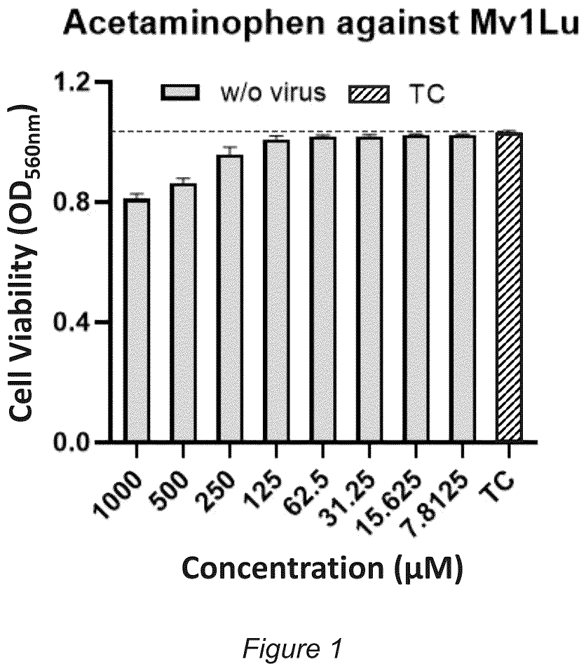

To maximize the possible effectiveness of acetaminophen, we tested the cytotoxicity of acetaminophen in vitro. Specifically, mink lung epithelial cells, Mv1Lu, were treated with acetaminophen. Before evaluating anti-CoV activity, we first tested the cytotoxicity of acetaminophen on both cell lines. As shown in FIG. 1, acetaminophen exhibited no significant cytotoxicity even though the concentration was elevated as high as 1000 μg/mL.

### In Vivo Test of Acetaminophen Against MHV-Infected Mice

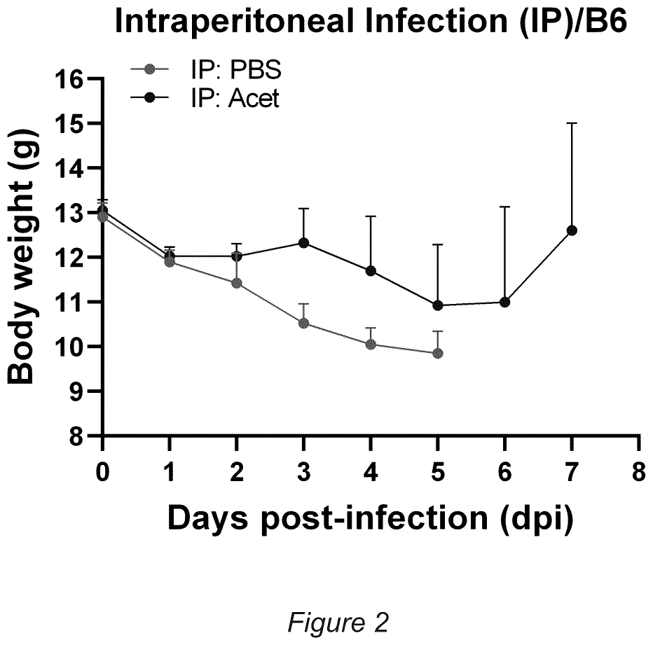

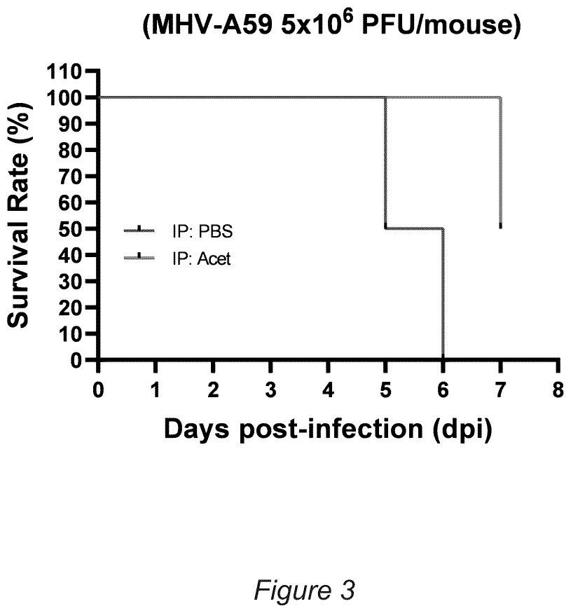

Intraperitoneal administration of MHV-A59 with high titer (5×10⁶ PFU/mouse) on C57BL/6 mice to cause a high mortality rate. The MHV-infected mice were grouped as follows: 1. PBS vehicle control; 2. 0.25 mg/mL of acetaminophen solution treatment groups followed by treating acetaminophen daily via oral gavage. The viral administration cause loss of body weights on mice. In terms of infected mice, acetaminophen significantly retained the mouse weights compared to MHV-only mice (FIG. 2). As shown in FIG. 3, comparing to 100% of death rate of the virus vehicle control, acetaminophen alone could elevate the survival rate to 50% of mortality rate, prolonging the devastating lethal challenge in animals. These animal data strongly argue the potential application of acetaminophen in developing to an antiviral agent.

### In Vivo Test of Acetaminophen Against PRRS-Infected Piglet

The PRRS virus (PRRSv) can affect all age groups. Reproductive impairment or failure is more obvious in sows or gilts, and the boars cannot. The respiratory syndrome is more often observed in young growing pigs but also occurs in naïve finishing pigs and older breeding stock. PRRS infects all types of herds including high or ordinary health status and both indoor and outdoor units, irrespective of size.

The PRRSv destroys up to 40 percent of the pig's macrophages removing a significant part of the pig's defending mechanism which allows secondary infections by bacteria and other viruses, particularly those that affect the respiratory system such as swine influenza, to proliferate and cause damage.

Clinical signs are evident as early as one week after weaning with the piglets losing condition with pale skin, mild coughing, sneezing and increased respiratory rates. Mortality during this period may reach 12-15 percent. Weaners and growers become vulnerable as their maternal antibody disappears and they then continually shed the virus for 3 to 8 weeks. These cases were alerting our research on the Indiscriminating anti-viral-drug.

In this embodiment, the weaned piglets were selected according to their PRRS syndromes. The three piglets were randomly selected in accordance with the professional experience on the syndromes of PRRS and the 5R principles of animal test were followed as well. Three infected piglets were proceeded for preliminary test of the effectiveness of antiviral drugs of PRRS.

Three infected piglets raised in an isolated pen of the same pig house. Eighty milliliter acetaminophen therapy was given three times orally. Their blood samples were test weekly. Unfortunately, piglet 1 was unable to make over the first week. The other 2 piglets were able make over the process. Piglets infected with the virus were treated with acetaminophen, piglets 2 and piglet 3 survived and the virus concentration in the body decreased. The laboratory results are listed in Table 1.

TABLE 1
Concentration of PRRSV2 were
conducted by RT-PCR

| | Piglet 1 | Piglet 2 | Piglet 3 |
|---|---|---|---|
| test 1 | 7.7 × 10 ⁵ | − | 8.2 × 10 ⁴ |
| test 2 | none | 1.3 × 10³ | ± |
| test 3 | none | − | 2.9 × 10 ² |

−: Negative
±: Virus was detected at very low concentrations

Based on the above results, in animal experiments, the drug can increase the survival rate and reduce the amount of virus against coronavirus, or PRRS virus, which has a great impact on piglets. It has a good therapeutic effect on animals infected with two different viruses. It is speculated that the drug can broadly treat most viral infections.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method of treating virus-associated infection in a subject, the method comprising:
administering a therapeutically effective amount of a pharmaceutical composition comprising an acetaminophen compound.

2. The method of claim 1, wherein the subject is experiencing coronavirus infection.

3. The method of claim 1, wherein the coronavirus is SARS-associated coronavirus.

4. The method of claim 1, wherein the subject is experiencing PRRS virus infection.

5. The method of claim 1, wherein the subject is a human or non-human mammal.

6. The method of claim 1, wherein the non-human mammal subject is a pig.

7. Use of acetaminophen for treatment or prevention of viral infections caused by virus.

8. The use as claimed in claim 7, wherein the virus is coronavirus.

9. The use as claimed in claim 8, wherein the coronavirus is SARS-associated coronavirus.

10. The use as claimed in claim 7, wherein the virus is PRRS virus.
